SEQUENCE LISTING (11 sequences)

SEQ ID NO: 1 - protein, 5 aa, synthetic construct
  source 1..5: note = MEDI3506 VHCDR1
SYAMS

SEQ ID NO: 2 - protein, 17 aa, synthetic construct
  source 1..17: note = MEDI3506 VHCDR2
GISAIDQSTYYADSVKG

SEQ ID NO: 3 - protein, 17 aa, synthetic construct
  source 1..17: note = MEDI3506 VHCDR3
QKFMQLWGGGLRYPFGY

SEQ ID NO: 4 - protein, 11 aa, synthetic construct
  source 1..11: note = MEDI3506 VLCDR1
SGEGMGDKYAA

SEQ ID NO: 5 - protein, 7 aa, synthetic construct
  source 1..7: note = MEDI3506 VLCDR2
RDTKRPS

SEQ ID NO: 6 - protein, 9 aa, synthetic construct
  source 1..9: note = MEDI3506 VLCDR3
GVIQDNTGV

SEQ ID NO: 7 - protein, 126 aa, synthetic construct
  source 1..126: note = MEDI3506 VH Domain
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSS

SEQ ID NO: 8 - protein, 106 aa, synthetic construct
  source 1..106: note = MEDI3506 VL Domain
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVL

SEQ ID NO: 9 - protein, 212 aa, synthetic construct
  source 1..212: note = MEDI3506 light chain
SYVLTQPPSVSVSPGQTASITCSGEGMGDKYAAWYQQKPGQSPVLVIYRDTKRPSGIPERFSGSNSGNTATLTISGTQAMDEADYYCGVIQDNTGVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - protein, 456 aa, synthetic construct
  source 1..456: note = MEDI3506 heavy chain
EVQLLESGGGLVQPGGSLRLSCAASGFTFSSYAMSWVRQAPGKGLEWVSGISAIDQSTYYADSVKGRFTISRDNSKNTLYLQMNSLRAEDTAVYYCARQKFMQLWGGGLRYPFGYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 260 aa, Homo sapiens
  source 1..260: note = IL-33 full length protein
MKPKMKYSTNKISTAKWKNTASKALCFKLGKSQQKAKEVCPMYFMKLRSGLMIKKEACYFRRETTKRPSLKTGRKHKRHLVLAACQQQSTVECFAFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCT

(19) United States
(12) Patent Application Publication
(10) Pub. No.: US 2026/0347458 A1
(43) Pub. Date: Nov. 5, 2026

(54) TREATMENT OF ACUTE RESPIRATORY FAILURE

(71) Applicant: MEDIMMUNE LIMITED, Cambridge (GB)

(72) Inventors: Hitesh Champaklal PANDYA, Cambridge (GB); Andrew Martin JONES, Wilmington, DE (US); Martin JENKINS, Cambridge (GB); Natalya MAKULOVA, Wilmington, DE (US); Christopher Martin KELL, Cambridge (GB); Ioannis PSALLIDAS, Cambridge (GB)

(21) Appl. No.: 19/104,870

(22) PCT Filed: Aug. 18, 2023
(86) PCT No.: PCT/EP2023/072809

§ 371 Date: Feb. 19, 2025

Related U.S. Application Data
(60) Provisional application No. 63/371,936, filed on Aug. 19, 2022.

(30) Foreign Application Priority Data
Sep. 23, 2022 (GB) 2213964.6

(51) Int. Cl. C07K 16/24; A61K 39/00; A61P 11/00; A61P 31/14

(52) U.S. Cl. CPC C07K 16/244; A61P 11/00; A61P 31/14; A61K 2039/505; A61K 2039/54

(57) ABSTRACT

The present disclosure provides a method of treating or preventing acute respiratory distress syndrome (ARDS) in a subject suffering from or at risk of developing ARDS, such as a subject with viral lung infection requiring supplemental oxygen. Said methods comprise administering to the subject a dose of 250 to 350 mg of an anti-IL-33 antibody. The methods may further comprise preventing progression of the subject to invasive mechanical ventilation (IMV) or extracorporeal membrane oxygenation (ECMO).

### CROSS-REFERENCE TO RELATED APPLICATIONS

This application claims benefit under 35 U.S.C. § 119(e) of U.S. Provisional Patent Application No. 63/371,936, filed Aug. 19, 2022, and benefit of GB Patent Application No. 2213964.6, filed Sep. 23, 2022, and which are incorporated by reference herein in their entirety for all purposes.

### REFERENCE TO SEQUENCE LISTING SUBMITTED ELECTRONICALLY

This application incorporates by reference a Sequence Listing submitted with this application in computer readable form (CRF) as a text file entitled “IL33-440-WO-PCT Sequence Listing” created on Aug. 17, 2023 and having a size of 13,464 bytes.

### FIELD

The present disclosure relates to the treatment of acute respiratory failure with anti-IL33 antibodies, in particular tozorakimab.

### BACKGROUND

Acute respiratory viral diseases are of major global public health importance and continue to cause over 1.5 million deaths a year. The immune-mediated damage resulting from dysregulated inflammatory responses leading to development of acute respiratory distress syndrome (ARDS) is a major contributor to severity of lung injury and unfavourable prognosis in respiratory viral disease. Over the past decades, several pandemic respiratory viruses, including influenza A H1N1 and H5N1 and the novel coronaviruses MERS-CoV, SARS-CoV, SARS-CoV-2 caused significantly higher frequency of ARDS and mortality compared to seasonal viruses. The COVID-19 pandemic has caused over 6 million deaths (as of September 2022), and escalated the imperative for development of new effective treatments to prevent and treat viral induced ARDS and/or acute respiratory failure (ARF).

WO 2021/204707 discloses the treatment and prevention of ARDS using an IL-33 antagonist, including anti-IL-33 antibodies. Tozorakimab was included in the ACCORD-2 phase II clinical trial for COVID-19 treatment (Wilkinson et al., Trials 21: 691, 2020).

### SUMMARY

In a first aspect, the disclosure provides a method of treating or preventing acute respiratory distress syndrome (ARDS) in a subject suffering from or at risk of developing ARDS, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a second aspect, the disclosure provides a method of treating or preventing acute respiratory failure (ARF) in a subject suffering from or at risk of developing ARF, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a third aspect, the disclosure provides a method of treatment to reduce the risk of a subject requiring invasive mechanical ventilation (IMV) or extracorporeal membrane oxygenation (ECMO), wherein the subject has or is at risk of developing ARDS and/or ARF, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a fourth aspect the disclosure provides a method of treatment to reduce the risk of a subject with or at risk of developing ARDS and/or ARF requiring admission to an intensive care unit (ICU), the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a fifth aspect the disclosure provides a method of treatment for reducing the duration of hospitalisation of a subject with, or at risk of developing, ARDS and/or ARF, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a sixth aspect the disclosure provides a method of treatment of a subject with or at risk of developing ARDS and/or ARF and who requires supplemental oxygen therapy, which treatment reduces the required duration of the supplemental oxygen therapy, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a seventh aspect, the disclosure provides a method of treatment for preventing or reducing the risk of respiratory failure in a subject hospitalised with a viral lung infection or suspected viral lung infection, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In an eighth aspect the disclosure provides a method of treating a subject hospitalised with a viral lung infection or suspected viral lung infection, the method comprising administering to the subject a dose of 250 to 350 mg of an anti-IL-33 antibody, wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a related aspect the disclosure provides an anti-IL-33 antibody for use in the treatment or prevention of ARDS or ARF in a subject, wherein the antibody, treatment or prevention, ARDS, ARF and/or subject are as defined above.

In a related aspect the disclosure provides the use of an anti-IL-33 antibody, in the manufacture of a medicament for the treatment or prevention of ARDS or ARF in a subject, wherein the antibody, treatment or prevention, ARDS, ARF and/or subject are as defined above.

In a related aspect the disclosure provides a pharmaceutical composition comprising an anti-IL-33 antibody, for use in the treatment or prevention of ARDS or ARF in a subject, wherein the antibody, treatment or prevention, ARDS, ARF and/or subject are as defined above.

In a related aspect, the disclosure provides an anti-IL-33 antibody, for use in the treatment or prevention of respiratory failure in a subject hospitalised with a viral lung infection or suspected viral lung infection, wherein said treatment comprises administering to the subject a dose of 250 to 350 mg of the anti-IL-33 antibody, and wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

In a related aspect, the disclosure provides an anti-IL-33 antibody, for use in the treatment of a viral lung infection or suspected viral lung infection in a subject, wherein the subject is hospitalised with the viral lung infection or suspected viral lung infection, wherein said treatment comprises administering to the subject a dose of 250 to 350 mg of the anti-IL-33 antibody, and wherein the antibody comprises:
(a) a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and
(b) a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6.

### DETAILED DESCRIPTION

The present disclosure relates to the use of an anti-IL-33 antibody in the treatment or prevention of diseases and conditions including acute respiratory distress syndrome (ARDS), acute respiratory failure (ARF) and diagnosed or suspected viral lung infections.

The term “IL-33” as employed herein refers to interleukin 33, in particular a mammalian interleukin-33 protein, generally the human IL-33 protein with the UniProt accession number 095760. This entity is not a single species but instead exists in several forms with different functional activities e.g. full length and proteolytically processed forms or oxidized and reduced forms. Given the rapid oxidation of the reduced form in vivo, and in vitro, generally prior art references to IL-33 might be most relevant to detection of the oxidized form. The terms “IL-33” and “IL-33 polypeptide” and “IL-33 protein” are used interchangeably herein.

IL-33 is a pleiotropic nuclear alarmin cytokine from the IL-1 superfamily. A full-length, reduced form of IL-33 (IL-33^{red}) is released from damaged epithelial and endothelial barrier cells and alerts the immune system to tissue damage. IL-33 drives pulmonary inflammation through its receptor ST2, which is expressed by several inflammatory cell types including mast cells, type 1 and 2 innate lymphoid cells, macrophages and endothelial cells. The IL-33/ST2 signaling pathway leads to production of inflammatory cytokines such as IL-6 and granulocyte-macrophage colony-stimulating factor by these cell types.

IL-33 is known to be released in response to multiple viral pathogens that are collectively responsible for the majority of severe viral lung and lower airway infection, including influenza, RSV, HRV, and SARS-CoV-2. Animal models of acute and chronic lung injury are similarly associated with elevated IL-33 and upregulation of type 1/2 cytokines (e.g. IL-6) and preclinical studies show IL-33 blockade can attenuate inflammation and improve lung function and symptoms (Allinne et al., J Allergy Clin Immunol. 2019,144(6):1624-37.e10). IL-33 is released by pulmonary epithelial cells infected with human respiratory viruses.

The term “antibody” is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

In some instances, the antibody used herein may be a monoclonal antibody (MAb); recombinant; chimeric; human; an antibody variant, including single chain; and/or bispecific; or derivatives thereof. Antibody antigen-binding fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. “Monoclonal antibody” or “monoclonal antibody composition” as used herein refers to polypeptides, including antibodies, bispecific antibodies, etc., that have substantially identical amino acid sequence or are derived from the same genetic source. This term also includes preparations of antibody molecules of single molecular composition. A monoclonal antibody composition displays a single binding specificity and affinity for a particular epitope. One instance is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

Suitably, a full-length antibody is used herein (that is to say, not an antibody fragment or derivative). Suitably the antibody used herein is a monoclonal antibody. Suitably the antibody used herein is human. Suitably a human monoclonal antibody is used.

The antibody used herein suitably comprises a heavy chain variable region comprising a VHCDR1 comprising the sequence of SEQ ID NO: 1, a VHCDR2 comprising the sequence of SEQ ID NO: 2, and a VHCDR3 comprising the sequence of SEQ ID NO: 3; and a light chain variable region comprising a VLCDR1 comprising the sequence of SEQ ID NO: 4, a VLCDR2 comprising the sequence of SEQ ID NO: 5, and a VLCDR3 comprising the sequence of SEQ ID NO: 6. However, in some instances the CDR sequences may be modified or altered relative to those defined in SEQ ID NOs: 1-6. For instance, VHCDR1, VHCDR2, VHCDR3, VLCDR1, VLCDR2 and/or VLCDR3 may comprise sequences modified by 1 to 3 amino acid substitutions, deletions and/or additions relative to SEQ ID NOs: 1-6, respectively.

The antibody used herein may comprise a heavy chain variable region comprising the sequence set forth in SEQ ID NO: 7, or an amino acid sequence with at least 80, 85, 90 or 95% sequence identity thereto. When a heavy chain variable region is used which is modified relative to SEQ ID NO: 7, it is preferred that the heavy chain CDR sequences are as set out in SEQ ID NOs: 1-3, though they may be modified or altered as set out above.

The antibody used herein may comprise a light chain variable region comprising the sequence set forth in SEQ ID NO: 8, or an amino acid sequence with at least 80, 85, 90 or 95% sequence identity thereto. When a light chain variable region is used which is modified relative to SEQ ID NO: 8, it is preferred that the light chain CDR sequences are as set out in SEQ ID NOs: 4-6, though they may be modified or altered as set out above.

Suitably, the antibody or antigen-binding fragment comprises a heavy chain comprising the set forth in SEQ ID NO: 7, or an amino acid sequence with at least 80, 85, 90 or 95% sequence identity thereto; and a light chain variable region comprising the sequence set forth in SEQ ID NO: 8, or an amino acid sequence with at least 80, 85, 90 or 95% sequence identity thereto.

As used herein, the term “sequence identity” or “identity” denotes a property of sequences that measures their similarity or relationship. The term “sequence identity” or “identity” as used in the present disclosure means the percentage of pair-wise identical residues—following (homologous) alignment of a sequence of a protein or polypeptide of the disclosure with a sequence in question—with respect to the number of residues in the longer of these two sequences. Sequence identity is measured by dividing the number of identical amino acid residues by the total number of residues and multiplying the product by 100.

A skilled artisan will recognize available computer programs, for example BLAST (Altschul et al., Nucleic Acids Res, 1997), BLAST2 (Altschul et al., J Mol Biol, 1990), FASTA (which uses the method of Pearson and Lipman (1988)), the TBLASTN program, of Altschul et al. (1990) supra, GAP (Wisconsin GCG package, Accelerys Inc, San Diego USA) and Smith-Waterman (Smith and Waterman, J Mol Biol, 1981), for determining sequence identity using standard parameters. The percentage of sequence identity can, for example, be determined herein using the program BLASTP, version 2.2.5, Nov. 16, 2002 (Altschul et al., Nucleic Acids Res, 1997). In this instance, the percentage of homology is based on the alignment of the entire protein or polypeptide sequences (matrix: BLOSUM 62; gap costs: 11.1; cut off value set to 10⁴) including the polypeptide sequences, suitably using the wild-type protein scaffold as reference in a pairwise comparison. It is calculated as the percentage of numbers of “positives” (homologous amino acids) indicated as result in the BLASTP program output divided by the total number of amino acids selected by the program for the alignment. Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps, which are spaces in an alignment that are the result of additions or deletions of amino acids. Generally, default parameters are used, with a gap creation penalty equaling 12 and a gap extension penalty equaling 4.

Specifically, in order to determine whether an amino acid residue of the amino acid sequence of an anti-IL-33 antibody is different from another antibody sequence, a skilled artisan can use means and methods well-known in the art, e.g., alignments, either manually or by using computer programs such as BLAST 2.0, which stands for Basic Local Alignment Search Tool, or Clustal Omega, or any other suitable program which is suitable to generate sequence alignments.

When a full-length antibody is used, it may be of any isotype or subclass thereof. Suitably the antibody is an IgG, e.g. IgG1, IgG2, IgG3 or IgG4 antibody. Suitably, the antibody is an IgG1.

Most suitably the antibody used in the therapies according to the disclosure is tozorakimab, as disclosed in WO2016/156440, which is incorporated herein by reference. Tozorakimab is also referred to in the art as MEDI3506 and 33_640087_7B. The light chain of tozorakimab has the amino acid sequence set forth in SEQ ID NO: 9, and the heavy chain of tozorakimab has the amino acid sequence set forth in SEQ ID NO: 10.

Tozorakimab is a fully human IgG1 monoclonal antibody that is being developed for the treatment of inter alia chronic obstructive pulmonary disease (COPD). Tozorakimab binds to the human reduced form of IL-33 (IL-33^{red}) and prevents binding of IL-33^{red} to its receptor, ST2. Tozorakimab binds human IL-33 with an exceptionally high affinity of approximately 30 fM, and fully neutralises full length and all mature forms of endogenous IL-33^{red} (Scott et al., ERS International Congress 2022, Barcelona (ES), Abstract OA2254). By binding to IL33^{red}, tozorakimab potently inhibits ST2-dependent inflammatory responses in several primary human cells, and in an allergen-driven in vivo model of lung epithelial injury. The oxidised form of IL-33 (IL-33^{ox}) which cannot bind ST2 signals via the RAGE/EGFR pathway. Tozorakimab cannot bind IL-33^{ox} but can prevent the oxidation of IL-33 and IL-33^{ox}-dependent signaling via the RAGE/EGFR complex and mimic the mechanism of action of ST2 (Scott et al., supra). Inhibition of IL-33^{ox} signaling by tozorakimab can improve airway epithelial repair functions and reverse airway epithelial dysfunction in respiratory diseases, including mucus hyper-secretion (Scott et al., supra).

In some instances the anti-IL-33 antibody has similar, or the same pharmacokinetic (pK) characteristics as tozorakimab in humans.

In particular, the anti-IL-33 antibody may have a similar, or the same, half-life in humans as tozorakimab. The anti-IL-33 antibody having a similar, or the same, half-life in humans as tozorakimab, when administered at a dose of 30 mg Q2W, may have a half-life of about 10 to about 20 days, about 12 to about 15 days, or of about 12.7 days. The anti-IL-33 antibody having a similar, or the same, half-life in humans as tozorakimab, when administered at a dose of 100 mg Q2W, may have a half-life of about 10 to about 20 days, about 12 to about 15 days, or of about 13.2 days. The anti-IL-33 antibody having a similar, or the same, half-life in humans as tozorakimab, when administered at a dose of 300 mg Q2W, may have a half-life of about 10 to about 20 days, about 12 to about 15 days, or of about 14.8 days.

In some instances, the IL-33 antibody may competitively inhibit binding of IL-33 to tozorakimab (tozorakimab is referred to as 33_640087-7B in WO2016/156440). WO2016/156440 discloses that 33_640087-7B (tozorakimab) binds to redIL-33 with particularly high affinity and attenuates both ST-2 and RAGE-dependent IL-33 signaling. An antibody is said to competitively inhibit binding of a reference antibody to a given epitope if it specifically binds to that epitope to the extent that it blocks, to some degree, binding of the reference antibody to the epitope. Competitive inhibition may be determined by any method known in the art, for example, solid phase assays such as competition ELISA assays, Dissociation-Enhanced Lanthanide Fluorescent Immunoassays (DELFIA®, Perkin Elmer), and radioligand binding assays. For example, the skilled person could determine whether an antibody competes for binding to IL-33 by using an in vitro competitive binding assay, such as the HTRF assay described in WO2016/156440, paragraphs 881-886, which is incorporated herein by reference. For example, the skilled person could label tozorakimab with a donor fluorophore and mix multiple concentrations with fixed concentration samples of acceptor fluorophore labelled-redIL-33. Subsequently, the fluorescence resonance energy transfer between the donor and acceptor fluorophore within each sample can be measured to ascertain binding characteristics. To elucidate competitive binding antibody molecules, the skilled person could first mix various concentrations of a test binding molecule with a fixed concentration of the labelled tozorakimab antibody. A reduction in the FRET signal when the mixture is incubated with labelled IL-33 in comparison with a labelled antibody-only positive control would indicate competitive binding to IL-33. An antibody may be said to competitively inhibit binding of the reference antibody to a given epitope by at least 90%, at least 80%, at least 70%, at least 60%, or at least 50%.

The therapies disclosed herein comprise administration of a dose of the anti-IL-33 antibody to the subject. The dose is in the range 250-350 mg (i.e. a flat dose rather than a body weight-dependent dose is used). In instances, the dose may be in the range 260-340 mg, 270-330 mg, 225-325 mg, 280-320 mg, 285-315 mg, 290-310 mg or 295-305 mg. Suitably the dose is 300 mg or about 300 mg. Such a dose of tozorakimab has been found to be effective in the reduction of respiratory failure or death in subjects hospitalised with COVID-19, as shown in the Examples below.

The dosage regimen utilised in the present disclosure may comprise administration of only a single dose of the antibody, or may comprise multiple doses (particularly two doses). In a particular instance the therapies of the present disclosure comprise administration of a single dose of the antibody to the subject. That is to say, the methods of treatment disclosed herein comprise administering a single dose of the antibody over a course of therapy.

When multiple doses of the antibody are administered, the doses are suitably spaced, i.e. a gap of an appropriate length is left between doses. For instance, a gap of at least a week, or two, three or four weeks, six weeks, or eight weeks, may be left between each dose. Generally, when multiple doses are administered in the present disclosure, each dose is of the same amount of antibody. In a particular instance, the therapies of the disclosure comprise administration of two doses of the antibody to the subject, wherein the second dose is administered at least a week after the first, suitably two weeks after the first. In some instances, the dosage regimen comprises administration of a first dose followed by optional administration of a second dose two weeks later, depending on the clinical condition/progress of the subject.

In one instance, the first dose of the antibody is administered after the subject has been hospitalised. In one instance, the first dose of the antibody is administered within 12, 24, 48 or 36 hours of hospitalization of the subject. In one instance, within 36 hours of hospitalization of the subject. Suitably hospitalisation of the subject may be regarded as admission to a hospital. In one instance, the first dose of the antibody is administered up to about 14 days after the onset of respiratory viral infection symptoms.

In one instance, the methods of treatment according to the disclosure comprise administration of a single dose. In an instance, the methods of treatment according to the disclosure comprise administration of a single, 300 mg dose of the antibody (tozorakimab) to the subject.

The antibody may be administered to the subject by any suitable route. Suitably, the antibody is administered intravenously.

In an instance, the methods of treatment according to the disclosure comprise intravenous administration of a single, 300 mg dose of the antibody (tozorakimab) to the subject.

In an instance, the methods of treatment according to the disclosure comprise intravenous administration of a single, 300 mg dose of the antibody (tozorakimab) to the subject within 36 hours of hospitalisation.

In an instance, the methods of treatment according to the disclosure comprise intravenous administration of a single, 300 mg dose of the antibody (tozorakimab) to the subject up to about 14 days after the onset of respiratory viral infection symptoms.

The antibody may be administered within a pharmaceutical composition. The pharmaceutical compositions may be formulated with suitable carriers, excipients, and other agents that provide suitable transfer, delivery, tolerance, and the like. A multitude of formulations can be found in the formulary known to all pharmaceutical chemists: Remington's Pharmaceutical Sciences, Mack Publishing Company, Easton, PA. Thus, the pharmaceutical compositions may comprise, in addition to the active ingredient (i.e. the anti-IL-33 antibody), a pharmaceutically acceptable excipient, carrier, buffer, stabiliser or other material well known to those skilled in the art. Such materials should be non-toxic and should not interfere with the efficacy of the active ingredient. The precise nature of the carrier or other material will depend on the route of administration, which may be by injection, e.g. intravenous or subcutaneous.

For intravenous injection, the pharmaceutical composition may be a parenterally acceptable aqueous solution which is pyrogen-free and has suitable pH, isotonicity and stability.

The pharmaceutical composition may be a liquid formulation or a lyophilized formulation which is reconstituted before use. As excipients for a lyophilized formulation, for example, sugar alcohols, or saccharides (e.g. mannitol or glucose) may be used. In the case of a liquid formulation, the pharmaceutical composition is usually provided in the form of containers with defined volume, including sealed and sterilized plastic or glass vials, ampoules and syringes, as well as in the form of large volume containers like bottles. Suitably, in the methods described herein, the pharmaceutical composition is a liquid formulation. Suitably, the liquid pharmaceutical composition is provided in a vial. Suitably, the anti-IL-33 antibody, may be present within the pharmaceutical composition at a concentration of from 100 mg/ml to 200 mg/ml, more suitably 150 mg/ml. In particular, when an antibody dose of 300 mg is used, the antibody (particularly tozorakimab) may be provided in 2 ml of a 150 mg/ml liquid composition.

Suitably, the anti-IL-33 antibody may be buffered to a pH of 5.2 to 5.7, most suitably 5.5 (e.g. ±0.1). The selection of such a pH confers significant stability to the pharmaceutical composition.

It will be appreciated that references to a “pharmaceutically acceptable excipient” includes references to any excipient conventionally used in pharmaceutical compositions. Such excipients may typically include one or more surfactant, inorganic or organic salt, stabilizer, diluent, solubilizer, reducing agent, antioxidant, chelating agent, preservative and the like.

Suitably, the surfactant is present within the pharmaceutical composition in an amount of from 0.001% to 0.1% (w/w). Suitably, the surfactant is polysorbate-80 (PS-80).

The anti-IL-33 antibody (particularly tozorakimab) may be provided in a pharmaceutical composition comprising L-histidine and/or L-histidine hydrochloride, L-arginine hydrochloride and polysorbate 80. The composition may in particular comprise 20 mM±10% L-histidine/L-histidine hydrochloride, e.g. 20 mM±2.5%, 5% or 7.5% L-histidine/L-histidine hydrochloride. That is to say L-histidine/L-histidine hydrochloride may be present in the composition at a concentration from 18-22, 18.5-21.5, 19-21 or 19.5-20.5 mM, suitably at a concentration of 20 mM.

The composition may in particular comprise 220 mM±10% L-arginine hydrochloride, e.g. 220 mM±2.5%, 5% or 7.5% L-arginine hydrochloride. For instance, L-arginine hydrochloride may be present in the composition at a concentration from 200-240, 205-235, 210-230 or 215-225 mM, suitably at a concentration of 220 mM.

The composition may in particular comprise 0.03% w/v±10% polysorbate 80, e.g. 0.03% w/v±2.5%, 5% or 7.5% polysorbate 80. For instance, polysorbate 80 may be present in the composition at a concentration from 0.027-0.033, 0.028-0.032 or 0.029-0.031% w/v, suitably at a concentration of 0.03% w/v.

The composition may have a pH from 5.2-5.7, 5.3-5.6 or 5.4-5.5, suitably 5.5.

In a particular instance the pharmaceutical composition comprises 20 mM L-histidine/L-histidine hydrochloride, 220 mM L-arginine hydrochloride and 0.03% polysorbate 80, and has a pH of 5.5. Suitably the pharmaceutical composition also comprises 150 mg/ml tozorakimab. When the composition comprises 150 mg/ml tozorakimab, a 300 mg dose of the antibody can be administered in 2 ml of the composition.

Following administration of the antibody to the subject, the subject may be administered saline solution, particularly a saline flush. When the antibody is administered intravenously, inclusion of a saline flush is preferred, in order to flush out the IV line. The saline solution is a sterile, physiological solution. Suitably the saline solution comprises 0.9% w/v±10% NaCl, e.g. 0.9% w/v±2.5%, 5% or 7.5% NaCl. Suitably the saline solution has a pH of 5.5±0.1. Suitably the saline solution comprises 0.9% w/v NaCl and has a pH of 5.5. The saline flush may have any suitable volume as necessary to flush the IV line, e.g. 2-10 ml, such as 2-8, 3-7 or 4-6 ml, suitably 5 ml.

Some aspects of the disclosure, as set out above, are directed to treating subjects suffering from or at risk of developing ARDS and/or ARF. Acute respiratory distress syndrome (ARDS) is a life-threatening condition where the lungs are unable to work properly. It is caused by injury to the capillary wall either from illness or a physical injury such as major trauma. This results in the wall becoming leaky, leading to a build-up of fluid and the eventual collapse of the air sacs, leaving the lungs unable to exchange oxygen and carbon dioxide. Acute respiratory failure (ARF) is a term often used alongside ARDS, but it is a broader term that refers to the failure of the lungs from any cause, e.g. chronic obstructive pulmonary disease (COPD).

Suitably a subject suffering from ARDS and/or ARF may be defined as a subject who is unable to ventilate adequately to provide sufficient oxygen to the blood and systemic organs.

Suitably the subject suffering from ARDS and/or ARF has one or more of the following symptoms: a higher than normal breathing rate, low blood oxygen concentration, laboured breathing, shortness of breath, low blood pressure, higher than normal heart rate, chest pain, skin colour changes, sweating, wheezing, confusion, and tiredness. Suitably ARDS and/or ARF may be defined as a subject who has at least one of the following symptoms: a higher than normal breathing rate, low blood oxygen concentration, laboured breathing, shortness of breath, and may additionally comprise one or more of the following symptoms: low blood pressure, higher than normal heart rate, chest pain, skin colour changes, sweating, wheezing, confusion, and tiredness.

Suitably a subject who is at risk of ARDS and/or ARF is likely to develop one or more of the following symptoms: a higher than normal breathing rate, low blood oxygen concentration, laboured breathing, shortness of breath, low blood pressure, higher than normal heart rate, chest pain, skin colour changes, sweating, wheezing, confusion, and tiredness. Suitably a subject who is at risk of ARDS and/or ARF may have few such symptoms, or no such symptoms, but may be at risk of developing further symptoms. Suitably a subject who is at risk of ARDS and/or ARF may have a disease, disorder, condition or infection as identified elsewhere herein which is associated with ARDS and/or ARF, or which is likely to lead to ARDS and/or ARF.

Suitably a subject suffering from ARDS and/or ARF may be a subject that requires oxygen or a subject that requires ventilation. In one instance, a subject suffering from ARDS and/or ARF is a subject that requires supplemental oxygen or ventilation. In one instance of any of the methods of treatment described herein, the subject requires supplemental oxygen or ventilation.

In one instance, the subject is suffering from acute respiratory failure (ARF), or is at risk of acute respiratory failure (ARF). In one instance, the subject is suffering from hypoxemic (Type 1) acute respiratory failure, or is at risk of hypoxemic (Type 1) acute respiratory failure. In one instance, the subject is suffering from hypercapnic (Type 2) acute respiratory failure, or is at risk of hypercapnic (Type 2) acute respiratory failure.

Suitably a subject who is at risk of ARDS and/or ARF, may be a subject having any one or more of the above symptoms, such as a higher than normal breathing rate, low blood oxygen concentration, laboured breathing, shortness of breath, low blood pressure, higher than normal heart rate, chest pain, skin colour changes, sweating, wheezing, confusion, and tiredness.

Suitably acute respiratory failure (ARF), may be caused by a condition, disease, disorder or infection, such as a bacterial or viral lung infection. Suitably the condition, disease, disorder or infection is a respiratory disease. Suitably the respiratory disease is a disease which affects the trachea, bronchi, bronchioles, alveolar ducts and/or alveoli.

Bacterial or viral respiratory infections associated with the ARDS and/or ARF may be selected from: tonsillitis, scarlet fever, pharyngitis, laryngitis, diphtheria, angina, Lemmiere syndrome, tularemia, the plague, enterocolitis, common cold, influenza, mononucleosis, HIV infection, pneumonia, suitably viral pneumonia, bronchitis, psittacosis, SARS, MERS, and COVID-19. In one instance, the viral respiratory infection is a viral lower respiratory infection or disease.

Suitably such infections may be caused by the following bacteria or viruses: Streptococcus sp., Arcanobacterium haemolyticum, Neisseria gonorrhoeae, Corynebacterium diphtheriae, Fusobacterium necrophorum, Francisella tulareniss, Yersinia pestis, Yersinia enterocolitica, Adenovirus sp., herpes simplex virus (HSV), HIV, Coxsackievirus sp., Coronavirus sp., Rhinovirus sp., Influenza A or B viruses, Parainfluenza viruses, Bocaparvovirus sp., Metapneumovirus sp., Respiratory syncytial virus (RSV), Epstein Barr virus, Cytomegalovirus sp., Mycoplasma pneumoniae, Chlamydophla pneumoniae, and Chhlmaydophla psittaci.

In one instance, the subject is suffering from ARDS and/or ARF caused by pneumonia, suitably viral pneumonia. In one instance, the subject is suffering from or has pneumonia, suitably viral pneumonia. In one instance, the subject at risk of pneumonia or viral pneumonia.

In one instance, the viral pneumonia is caused by COVID-19, suitably derived from infection with a Coronavirus which may be selected from any of those listed above, suitably from infection with SARS-CoV-2. In some instances, the pneumonia is caused by influenza virus A, influenza virus B, respiratory syncytial virus, human parainfluenza virus, adenovirus, metapneumovirus, SARS-COV, Middle East respiratory syndrome virus (MERS-CoV), hantavirus, herpes simplex virus, varicella-zoster virus, measles virus, rubella virus, cytomegalovirus, smallpox virus or dengue virus. In some instances, the pneumonia is caused by influenza virus A, influenza virus B, respiratory syncytial virus or human parainfluenza virus.

Suitably therefore, in some instances, the subject is suffering from, or has, both COVID-19 and viral pneumonia. In some instances wherein the viral pneumonia is caused by COVID-19, or a SARS-CoV-2 infection. In some instances, the subject is suffering from COVID-19 and is at risk of viral pneumonia.

The ARDS or ARF in the subject may have any cause, e.g. ARDS or ARF may be caused by pneumonia, chronic obstructive pulmonary disease (COPD), asthma, bronchitis, bronchiectasis, emphysema, heart failure, myocardial ischemia, mitral stenosis, pulmonary oedema, pulmonary embolism, thromboembolism, cystic fibrosis, amylotophic lateral sclerosis, muscular dystrophy, Guillain-Barre syndrome, myasthenia gravis, poliomyelitis, polymyositis, botulism, hypokalemia, hypophosphatemia, myxedema, hypothyroidism, sepsis, stroke, acute pancreatitis, transfusion, reperfusion, drug or alcohol overdose, trauma to the chest, viral or bacterial infection, inhalation injury, aspiration, and/or near drowning.

In a particular instance, the subject has (i.e. has been diagnosed with) or is suspected of having a viral lung infection (i.e. a viral infection of the lung). In a particular instance, the subject has (i.e. has been diagnosed with) or is suspected of having a viral lower respiratory tract infection or disease.

Diagnosis of a viral lung infection may be made by any means known in the art, e.g. a nucleic acid amplification test (e.g. using PCR or RT-PCR) or an antigen test (e.g. using a lateral flow test device). Herein “diagnosis” means the positive confirmation of a viral infection by testing, e.g. laboratory testing. A diagnosed viral lung infection can be contrasted with a suspected lung infection. A subject has a suspected lung infection if an examining physician believes that the subject has a lung infection (e.g. due to signs or symptoms upon presentation) but has not confirmed this by a diagnostic test, e.g. because test results are awaited, testing is unavailable or has failed, or when an infecting agent cannot be identified. Generally, when the subject has or is suspected of having a viral lung infection, the viral lung infection is the cause of the ARDS or ARF treated according to the disclosure, or the viral lung infection is putting the subject at risk of developing ARDS or ARF.

As noted above, in certain aspects of the disclosure, the subject has or is suspected of having a viral lung infection.

A viral lung infection may be caused by any known viral respiratory pathogen. For instance, the viral lung infection may be caused by a coronavirus, e.g. SARS-CoV, MERS-CoV or SARS-CoV-2 (the causative agent of COVID-19). In a particular instance, the viral lung infection is caused by SARS-CoV-2, i.e. the subject has COVID-19. In another instance, the viral lung infection is not caused by SARS-CoV-2, i.e. a virus other than SARS-CoV-2 is the causative agent of the infection.

In another particular instance of the disclosure, the viral lung infection is caused by an influenza virus. Any influenza virus may be the cause, particularly an influenza A or influenza B virus. The influenza A virus may be a seasonal influenza subtype, e.g. a seasonal H1N1 or H3N2 subtype. Alternatively the influenza A virus may be a non-human (e.g. avian) strain or a pandemic strain, e.g. H5Nx (e.g. H5N1) or H7N9.

In another particular instance of the disclosure, the viral lung infection is caused by respiratory syncytial virus (RSV). In another instance the viral lung infection is caused by human metapneumovirus (HMPV).

In other instances, the viral lung infection is caused by human parainfluenza virus, adenovirus, hantavirus, herpes simplex virus, varicella-zoster virus, measles virus, rubella virus, cytomegalovirus, smallpox virus or dengue virus.

The subject may be hospitalised at the time the anti-IL-33 antibody is administered. That is to say, the subject may be in hospital. The hospitalisation is generally due to fact the subject has ARDS or ARF or a condition which puts them at risk of developing ARDS or ARF. Where the subject has or is suspected of having a viral lung infection, the subject is generally hospitalised due to the (suspected) viral lung infection (indeed some aspects of the disclosure are directed to the treatment of subjects hospitalised with a viral lung infection). In one instance, the subject is hospitalised.

In one instance of any of the methods described herein, the subject has, or is suspected of having, a viral lower respiratory tract infection, and optionally may be hospitalized, and/or optionally may require supplemental oxygen or ventilation. In one instance of any of the methods described herein, the subject has a viral lower respiratory tract infection, is hospitalized, and requires supplemental oxygen or ventilation.

When the subject is hospitalised due to ARDS or ARF or a condition which puts them at risk of developing ARDS or ARF (such as a viral lung infection) it is preferred that the anti-IL-33 antibody is administered within 36 hours of the subject's admission to hospital (that is to say the anti-IL-33 antibody is suitably administered to the subject no more than 36 hours after the subject is admitted to hospital). Suitably the anti-III-33 antibody is administered to the subject within 30, 24, 18, 12 or 6 hours of admission of the subject to hospital.

When the subject has a viral lung infection, and due to the infection is at risk of respiratory failure (e.g. ARDS or ARF), it is preferred that the anti-IL-33 antibody is administered to the subject up to about 14 days after the onset of symptoms of the viral infection. Early-stage symptoms of a viral respiratory infection are well known and include e.g. coughing, sneezing, sore throat and/or fever. Suitably the anti-IL-33 antibody is administered to the subject no more than 14 days after the onset of the first symptoms of the viral lung infection. In other instances the anti-IL-33 antibody is administered to the subject no more than 13, 12, 11, 10, 9, 8, 7, 6, 5, 4 or 3 days after onset of the first symptoms of the viral lung infection.

Commonly, the subject has hypoxaemia and so requires supplemental oxygen (i.e. oxygen therapy). Hypoxaemia is a condition whereby there is an oxygen deficiency in arterial blood. Hypoxaemia can be readily diagnosed by a physician. In particular instances, hypoxaemia is diagnosed when the subject: (i) has an SPO₂ (blood oxygen saturation) of 94% or less in room air (i.e. air with an atmospheric level of O₂, as opposed to oxygen-enriched air); (ii) is receiving oxygen therapy but had an SPO₂ of less than 94% prior to initiation of oxygen therapy; and/or (iii) is receiving at least (e.g. more than) 6 L/min of supplemental oxygen and/or non-invasive ventilation. In some instances, hypoxaemia is diagnosed when the subject has SpO₂≤90% OR SpO₂≤92% AND one or both of the following: (i) Radiographic infiltrates by Chest X-ray/CT scan compatible with viral lung infection per investigator judgement; or (ii) use of accessory muscles of respiration or respiratory rate >22/minute.

As will be understood from the above, the subject is suitably a human patient.

The therapies disclosed herein may reduce the risk of a subject at risk of respiratory failure, e.g. ARDS and/or ARF from developing respiratory failure, e.g. ARDS and/or ARF. For instance, the therapies disclosed herein may reduce the risk of such a subject developing respiratory failure, e.g. ARDS and/or ARF by at least 5, 10, 15, 20, 25, 30, 35, 40, 45 or 50%. Similarly, the therapies disclosed herein may reduce the risk of the subject developing pneumonia, e.g. by at least 5, 10, 15, 20, 25, 30, 35, 40, 45 or 50%. The therapies disclosed herein may reduce the risk of developing respiratory failure, ARDS, ARF and/or pneumonia by the recited amounts across a specified period following administration of the anti-IL-33 antibody (or the first dose of the anti-IL-33 antibody), e.g. across a period of 14, 28, 42, 56 or 60 days following administration of the anti-IL-33 antibody, particularly 28 days or 60 days following administration of the anti-IL-33 antibody.

The therapies disclosed herein may improve the survival prospects of a subject suffering from or at risk of developing respiratory failure, e.g. ARDS and/or ARF. For instance, the therapies disclosed herein may reduce the risk of such a subject dying from respiratory failure, e.g. ARDS or ARF by at least 5, 10, 15, 20, 25, 30, 35, 40, 45 or 50%. In particular, therapies disclosed herein may so reduce the risk of the subject dying from respiratory failure, e.g. ARDS or ARF across a period of e.g. 14, 28, 42, 56 or 60 days following administration of the anti-IL-33 antibody, particularly 28 days or 60 days following administration of the anti-IL-33 antibody.

That is to say, the therapies disclosed herein may treat or prevent respiratory failure, e.g. ARDS and/or ARF in a subject. In the context of a subject suffering from or at risk of respiratory failure due to a viral lung infection, the therapies disclosed herein may be seen as treating the viral lung infection. The therapies disclosed herein may also limit the severity of respiratory failure, e.g. ARDS and/or ARF in a subject, such that less hospital treatment or less intensive or invasive hospital treatment is required. In particular they may reduce the need for care in an intensive care unit (ICU), and/or reduce the need for invasive mechanical ventilation (IMV) or extracorporeal membrane oxygenation (ECMO).

In particular, the therapies disclosed herein may reduce the risk of a subject with or at risk of developing ARDS and/or ARF requiring IMV and/or ECMO by at least 5, 10, 15, 20, 25, 30, 35, 40, 45 or 50%. In particular, therapies disclosed herein may so reduce the risk of the subject requiring IMV and/or ECMO across a period of e.g. 14, 28, 42, 56 or 60 days following administration of the anti-IL-33 antibody, particularly 28 days or 60 days following administration of the anti-IL-33 antibody.

The therapies disclosed herein may reduce the risk of a subject with or at risk of developing ARDS and/or ARF requiring admission to an intensive care unit (ICU) by at least 5, 10, 15, 20, 25, 30, 35, 40, 45 or 50%. In particular, therapies disclosed herein may so reduce the risk of the subject requiring ICU admission across a period of e.g. 14, 28, 42, 56 or 60 days following administration of the anti-IL-33 antibody, particularly 28 days or 60 days following administration of the anti-IL-33 antibody.

If a subject with or at risk of developing ARDS and/or ARF requires admission to ICU, the therapies disclosed herein may reduce the duration of the required stay of the subject in ICU, in particular by at least 5, 10, 15, 20, 25, 30, 35, 40, 45 or 50%. Alternatively the therapies disclosed herein may reduce the duration of the required stay of the subject in ICU by at least 1, 2, 3, 4, 5, 6, 7, 8, 9 or 10 days. In this case, the required stay in ICU of the subject may be the average required stay in ICU of a group of subjects, all with or at risk of developing ARDS and/or ARF. Such a reduction in required ICU stay may be across a period of e.g. 14, 28, 42, 56 or 60 days following administration of the anti-IL-33 antibody, particularly 28 days or 60 days following administration of the anti-IL-33 antibody.

Similarly, the therapies disclosed herein may increase the number of days a subject with or at risk of developing ARDS and/or ARF is alive and outside ICU in the 60 days following administration of the anti-IL-33 antibody. The number of days the subject is alive and outside ICU for may be increased by at least 10, 20, 30, 40, 50, 60, 70, 80, 90 or 100% or more. Alternatively, the number of days the subject is alive and outside ICU for may be increased by at least 5, 10, 15, 20, 25, 30, 35, 40, 45, 50 or 55. In this case, the number of days the subject is alive and outside of ICU for may be the average number of days of a group of subjects, all with or at risk of developing ARDS and/or ARF.

The therapies disclosed herein may increase the number of days a subject with or at risk of developing ARDS and/or ARF is alive and does not require supplementary oxygen in the 60 days following administration of the anti-IL-33 antibody. The number of days the subject is alive and does not require supplementary oxygen for may be increased by at least 10, 20, 30, 40, 50, 60, 70, 80, 90 or 100% or more. Alternatively, the number of days the subject is alive and does not require supplementary oxygen for may be increased by at least 5, 10, 15, 20, 25, 30, 35, 40, 45, 50 or 55. In this case, the number of days the subject is alive and does not require supplementary oxygen for may be the average number of days of a group of subjects, all with or at risk of developing ARDS and/or ARF.

Relatedly, the therapies disclosed herein may reduce the required duration of supplemental oxygen therapy in a subject with or at risk of developing ARDS and/or ARF and who requires supplemental oxygen therapy, in particular by at least 5, 10, 15, 20, 25, 30, 35, 40, 45 or 50%. Alternatively the therapies disclosed herein may reduce the duration of the required supplemental oxygen therapy by at least 2, 4, 6, 8, 10, 12, 14, 16, 18 or 20 days. In this case, the required duration of supplemental oxygen therapy of the subject may be the average required duration of oxygen therapy of a group of subjects, all with or at risk of developing ARDS and/or ARF. Such a reduction in required duration of supplemental oxygen therapy may be across a period of e.g. 14, 28, 42, 56 or 60 days following administration of the anti-IL-33 antibody, particularly 28 days or 60 days following administration of the anti-IL-33 antibody.

As used herein, the terms “oxygen therapy”, “supplemental oxygen” and “supplemental oxygen therapy” are interchangeable.

The therapies disclosed herein may reduce the required duration of hospitalisation in a subject with or at risk of developing ARDS and/or ARF, in particular by at least 5, 10, 15, 20, 25, 30, 35, 40, 45 or 50%. Alternatively the therapies disclosed herein may reduce the duration of the required hospitalisation by at least 2, 4, 6, 8, 10, 12, 14, 16, 18 or 20 days. In this case, the required duration of hospitalisation of the subject may be the average required duration of hospitalisation of a group of subjects, all with or at risk of developing ARDS and/or ARF. Such a reduction in required duration of hospitalisation may be across a period of e.g. 14, 28, 42, 56 or 60 days following administration of the anti-IL-33 antibody, particularly 28 days or 60 days following administration of the anti-IL-33 antibody.

The therapies disclosed herein may increase the chance of a subject with or at risk of developing ARDS and/or ARF being discharged from hospital alive, e.g. by at least 10, 20, 30, 40, 50, 60, 70, 80, 90 or 100% or more. Such an increase in the chance of discharge of the subject from hospital alive may be across a period of e.g. 14, 28, 42, 56 or 60 days following administration of the anti-IL-33 antibody, particularly 28 days or 60 days following administration of the anti-IL-33 antibody.

The therapies disclosed herein may reduce the risk of a subject with or at risk of developing ARDS and/or ARF, who is initially discharged from hospital alive, requiring readmission to hospital within 28 or 60 days of administration of the anti-IL-33 antibody. Such a risk may be reduced by e.g. at least 5, 10, 15, 20, 25, 30, 35, 40, 45 or 50%.

Where the subject has a viral lung infection caused by SARS-CoV-2 (i.e. the subject has COVID-19) the therapies disclosed herein may reduce or prevent disease progression as defined by the WHO 10-point clinical progression scale for COVID-19, or cause an improvement in the condition of the subject as defined by said clinical progression scale. For example, the treatment may increase the chance of the subject having a score less than 7, 6, 5, 4 or 3 as defined by the WHO 10-point clinical progression scale for COVID-19 at day 28 or 60 following administration of the anti-IL-33 antibody, or cause the subject to reach a score of less than 7, 6, 5, 4 or 3 according to said scale more quickly. The WHO 10-point clinical progression scale for COVID-19 is set out below:

| Score | Descriptor |
|---|---|
| 0 | Uninfected; no viral RNA detected |
| 1 | Asymptomatic; viral RNA detected |
| 2 | Symptomatic; independent |
| 3 | Symptomatic; assistance needed |
| 4 | Hospitalised; no oxygen therapy |
| 5 | Hospitalised; oxygen by mask or nasal prongs |
| 6 | Hospitalised; oxygen by NIV or high flow |
| 7 | Intubation and mechanical ventilation, |
| | pO2/FiO2 ≥150 or SpO2/FiO2 ≥200 |
| 8 | Mechanical ventilation pO2/FIO2 <150 |
| | (SpO2/FiO2 <200) or vasopressors |
| 9 | Mechanical ventilation pO2/FiO2 <150 and |
| | vasopressors, dialysis, or ECMO |
| 10 | Dead |

Where treatment with the IL-33 antibody reduces risks associated with or improves recovery from ARDS/ARF in the manners set out above, the reductions or improvements are made in comparison to a control patient population with the same condition as the subject but to whom an anti-IL-33 antibody (or fragment thereof) is not administered. The control patient population receives the standard of care for the condition with the exception of the anti-IL-33 antibody. That is to say the control patient population receives the same medical care as the subject, except for the anti-IL-33 antibody. The improvements in clinical outcomes described above are therefore the direct result of administration of the antibody (or fragment thereof) to the subject.

The disclosure can be further understood by the following non-limiting examples and figures.

### FIGURE LEGENDS

FIG. 1—Study design of Phase III, Multicentre, Randomised, Double-blind, Parallel-group, Placebo-controlled Study to Evaluate the Efficacy and Safety of Tozorakimab (MEDI3506) in Patients Hospitalised for Viral Lung Infection Requiring Supplemental Oxygen (TILIA).

### EXAMPLES

### Example 1—ACCORD2: a Multicentre, Seamless, Phase 2 Adaptive Randomisation Platform Study to Assess the Efficacy and Safety of Multiple Candidate Agents for the Treatment of COVID-19 in Hospitalised Patients

### Study Design

The objectives of the study were to assess the efficacy and safety of tozorakimab 300 mg IV plus SoC, compared with SoC alone, in hospitalised adults with SARS-CoV-2 (COVID-19) infection with an WHO 8-point ordinal clinical progression scale score of 3, 4, or 5 (See Table 1).

In the study, MEDI3506 (tozorakimab) was administered to patients as a single 300 mg IV dose. A second dose of 300 mg IV tozorakimab was administered if the patient became invasively ventilated on, or before, Day 15 but after randomisation and remained invasively ventilated on Day 15. A dose of 300 mg tozorakimab IV is predicted to have a safety margin of >47-fold for both maximum observed concentration and area under the concentration-time curve (AUC) over exposures at the no observed adverse effect level (150 mg/kg) based on a 4-week Good Laboratory Practice toxicology study.

In total, 103 patients were randomised to either tozorakimab plus Standard of Care (SoC) (n=56) or SoC alone (n=47). Three patients on SoC alone were randomised after the tozorakimab sub-study had closed, and 2 patients were randomised to tozorakimab plus SoC but were not dosed; these patients were excluded from the Safety Analysis and Full Analysis Sets. One patient received a dose of tozorakimab but did not have any post-baseline ordinal scale data, and therefore was excluded from the Full Analysis Set. In total, 98 patients in the Safety Analysis Set received either tozorakimab plus SoC (n=54) or SoC alone (n=44).

In the Safety Analysis Set, demographic characteristics were well-balanced across treatment arms. The majority of patients were male (for tozorakimab plus SoC vs SoC alone: 37 patients [68.5%] vs 29 patients [65.9%]) and middle aged or older (mean [SD] for tozorakimab plus SoC vs SoC alone: 55.4 years [12.51] vs 58.0 years [13.90]). A higher percentage of patients had diabetes and 2 co-morbidities in the tozorakimab plus SoC arm (diabetes: 22 patients [40.7%]; 2 co-morbidities: 21 patients [38.9%]) compared with the SoC alone arm (diabetes: 13 patients [29.5%]; 2 co-morbidities: 13 patients [29.5%]). Standard of care evolved during the study treatment period, with increased use of dexamethasone and remdesivir as well as the addition of tocilizumab later on in the study. The primary endpoint of time to sustained clinical response was defined as a 2-point improvement on the ordinal scale, discharged from hospital, or considered fit for discharge, whichever came first, by Day 29. The WHO 8-point ordinal Clinical Progression Scale was used, as recommended by the WHO at the time the study was designed (Table 1).

TABLE 1
WHO 8-point Ordinal Clinical Progression Scale, Source: (WHO, 2020)

| Score | Description |
|---|---|
| 0 | Uninfected, no clinical or virological evidence of infection |
| 1 | Ambulatory, no limitation of activities |
| 2 | Ambulatory, limitation of activities |
| 3 | Hospitalised-mild disease, no oxygen therapy |
| 4 | Hospitalised-mild disease, oxygen by mask or nasal prongs |
| 5 | Hospitalised-severe disease, non-invasive ventilation or high- |
| | flow oxygen |
| 6 | Hospitalised-severe disease, intubation and mechanical |
| | ventilation |
| 7 | Hospitalised-severe disease, ventilation and additional organ |
| | support-vasopressors, renal replacement therapy, |
| | extracorporeal membrane oxygenation |
| 8 | Death |

### Study Results

### Primary Endpoint

No statistically significant difference between treatment arms was observed based on the hazard ratio for time to sustained clinical response (Table 2).

The benefit of tozorakimab was observed not on speed of discharge as many patients were able to be successfully treated quickly, but rather on the prevention of morbidity or mortality in those patients who could not be promptly discharged (see “Key Secondary Endpoints” below).

TABLE 2
Time to Sustained Clinical Response by Day 29 (Full Analysis Set)

| | Tozorakimab | |
|---|---|---|
| | plus SoC | SoC alone |
| Parameter | (N = 53) | (N = 44) |
| Patients censored, n (%) | 11 (20.8) | 12 (27.3) |
| Median time to response, | 8 (7.00, 9.00) | 9.5 (7.00, 13.00) |
| days (80% CI) | | |
| Hazard ratio (80% CI)^{c} | 0.96 (0.700, 1.306) | NA |
| One-sided p-value | 0.4267 | NA |

^{a}Patients who died prior to Day 29, or who did not respond by Day 29, were censored at Day 29.
^{b}Kaplan-Meier product-limit estimate provided, with 80% CI calculated according to Brookmeyer and Crowley.
^{c}Hazard ratio computed from Cox-proportional hazard model after adjusting for age, treatment, and binary baseline ordinal score as covariates. A Hazard Ratio > 1 indicates a treatment effect in favour of tozorakimab.
CI = confidence interval.

### Key Secondary Endpoints

For the secondary endpoint of death or respiratory failure by Day 29 respiratory failure was defined as a score of 6 or 7 on the ordinal scale. Patients in the tozorakimab plus SoC arm had lower odds of death or respiratory failure by Day 29 compared with SoC alone (Table 3).

Patients in the tozorakimab arm had lower odds of mortality by Day 29 in the tozorakimab plus SoC arm compared with SoC alone (Table 4).

In relative terms there was around a one third reduction in risk for both endpoints in patients. Although neither finding represented a statistically significant difference this was a small pilot study to identify potential treatments and was not prospectively powered for these endpoints. These findings suggest the potential for clinically meaningful benefits with tozorakimab and indicate that further study is justified.

TABLE 3
Death or Respiratory Failure by Day 29 (Full Analysis Set)

| | Tozorakimab plus | SoC |
|---|---|---|
| Parameter | SoC (N = 53) | (N = 44) |
| Death or respiratory failure by Day 29 | | |
| Yes, n (%) | 9 (17.0%) | 11 (25.0%) |
| No, n (%) | 44 (83.0%) | 33 (75.0%) |
| Odds ratio (80% CI) | 0.55 (0.272, 1.123) | NA |
| P-value | 0.2835 | NA |

^{a}Calculated from logistic regression model adjusting for age and baseline severity.
CI = confidence interval;
NA = not applicable;
SoC = standard of care.

TABLE 4
Mortality by Day 29 (Full Analysis Set)

| | Tozorakimab plus | SoC |
|---|---|---|
| Parameter | SoC (N = 53) | (N = 44) |
| Death by Day 29 | | |
| Yes, n (%) | 6 (11.3%) | 6 (13.6%) |
| No, n (%) | 47 (88.7%) | 38 (86.4%) |
| Odds ratio (80% CI) | 0.70 (0.286, 1.709) | NA |
| P-value | 0.6074 | NA |

^{a}Calculated from logistic regression model adjusting for age and baseline severity.
CI = confidence interval;
NA = not applicable;
SoC = standard of care.

### Example 2—A Phase III, Multicentre, Randomised, Double-Blind, Parallel-Group, Placebo-Controlled Study to Evaluate the Efficacy and Safety of Tozorakimab (MED13506) in Patients Hospitalised or Viral Lung Infection Requiring Supplemental Oxygen (TILIA)

### Overall Study Design

This is a Phase III, multicentre, randomised, double-blind, parallel-group, placebo-controlled study to evaluate the efficacy and safety of tozorakimab in patients hospitalised for viral lung infection requiring supplemental oxygen to reduce risk of progression to acute respiratory distress syndrome or death.

The primary outcome is the proportion of participants who die or progress to Invasive Mechanical Ventilation (IMV)/Extracorporeal Membrane Oxygenation (ECMO) by Day 28. Study intervention will be administered on Day 1. Patient status will be recorded daily while in hospital. Upon discharge, the participants will be followed up by phone at Day 14 and Day 28. Final on-site visit will be performed on Day 60.

The study initially plans to randomise approximately 2352 participants (i.e, 1176 per treatment arm), although the final sample size will be determined by the number of events. Randomisation will be stratified by known viral positivity at randomisation (SARS-CoV-2 versus other virus versus indeterminate), and region. The study will recruit until approximately 375 primary endpoint events have been observed in the participants with confirmed viral positivity from baseline samples (known prior or after randomisation). At least 60% of participants are expected to have confirmed positive viral test at randomisation with the intent to have at least approximately 75% of viral positive cases overall (including cases confirmed retrospectively) in the study population. Participants will be randomised in a 1:1 ratio to receive tozorakimab 300 mg or matching placebo, administered intravenously (IV injection) within 36 hours from the admission to hospital.

Participants will receive a single dose of study intervention (tozorakimab 300 mg or placebo) administered by IV injection followed by 5 mL saline flush.

Participants' vital signs and WHO Clinical Progression Scale will be assessed and recorded once daily during hospitalisation.

The study intervention will be administered on top of SoC treatments. Study participants will continue receiving SoC based on local guidelines throughout the study.

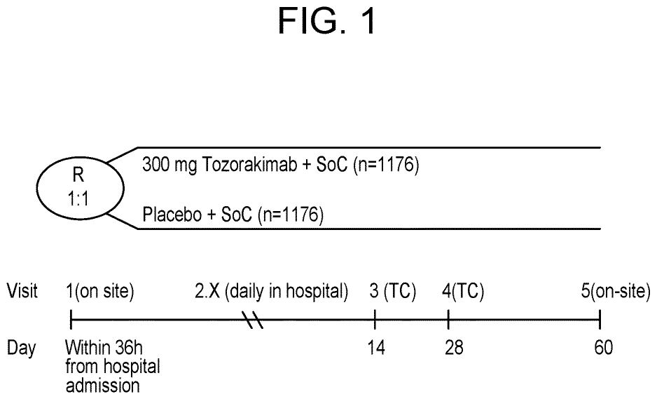

FIG. 1 shows a schematic representation of the study design.

### Justification for Dose

Tozorakimab will be administered to participants as a single 300 mg IV injection, followed by 5 mL saline flush.

The nonclinical and clinical safety and efficacy data generated with tozorakimab provides a positive risk/benefit ratio for the clinical program in adults hospitalised with acute viral infection requiring supplemental oxygen and at risk to develop ARDS.

On top of SoC, this dose and route of administration was tested in ACCORD-2 study in patients with COVID-19 and showed a numerical reduction of 32% in the proportion of participants who had died or had experienced respiratory failure by Day 29, versus SoC alone (Example 1). In this study, there were no safety findings that preclude further development of this drug. Also, single 300 mg IV injection of tozorakimab was the highest dose tested in the first time in man single ascending dose study conducted in healthy participants with mild history of atopy with good safety and tolerability profile.

### Objectives, Endpoints, and Estimands

Objectives and endpoints are presented in Table 5.

TABLE 5
Objectives and endpoints

| | |
|---|---|
| Objectives | Endpoints |

Primary

| | |
|---|---|
| To evaluate the effect of tozorakimab | Primary Endpoint: Proportion of |
| versus placebo as add on to SoC in | participants who die or progress to |
| participants with viral lung infection | IMV/ECMO by Day 28 |
| requiring supplemental oxygen on the | Population^{a}: Confirmed Viral Positive |
| prevention of death or progression to | Analysis Set |
| IMV/ECMO by Day 28 | Intercurrent Events^{b}: Death (all cause) |
| | incorporated into composite outcome. |
| | Treatment policy otherwise. All available |
| | data from participants will be included up to |
| | Day 60. |
| | Summary Measure: Relative risk ratio |

Key secondary

| | |
|---|---|
| To evaluate the effect of tozorakimab | Key Secondary Endpoint: Proportion of |
| versus placebo as add-on to SoC on all- | participants who die by Day 60 |
| cause mortality by Day 60 | Population ^{a}: Confirmed Viral Positive |
| | Analysis Set |
| | Intercurrent Events ^{b}: Death (all cause) |
| | incorporated into composite outcome. |
| | Treatment policy otherwise. |
| To evaluate the effect of tozorakimab | Key Secondary Endpoint: Number of |
| versus placebo as add-on to SoC on ICU | days alive and outside of ICU over 28 day |
| stay | period |
| | Population ^{a}: Confirmed Viral Positive |
| | Analysis Set |
| | Intercurrent Events ^{b}: Death (all cause) |
| | incorporated into composite outcome. |
| | Treatment policy otherwise. |
| | Summary Measure: Difference in medians |
| | (rank based comparison) |
| To evaluate the effect of tozorakimab | Key Secondary Endpoint: Number of days |
| versus placebo as add-on to SoC on the | alive and free of supplemental oxygen over 28 |
| duration of oxygen supplementation | day period |
| | Population ^{a}: Confirmed Viral Positive Analysis |
| | Set |
| | Intercurrent Events ^{b}: Death (all cause) |
| | incorporated into composite outcome. |
| | Treatment policy otherwise. |
| | Summary Measure: Difference in medians |
| | (rank based comparison) |

Secondary ^{c}

| | |
|---|---|
| To evaluate the effect of tozorakimab | Time to death or progression to IMV/ECMO |
| versus placebo as add-on to SoC on | Proportion of participants who die or |
| prolonging time to death or IMV/ECMO | progress to IMV/ECMO by Day 60 |
| To evaluate the effect of tozorakimab | Time to Death (all cause) |
| versus Placebo as add-on to SoC on | Proportion of participants who die by Day |
| prolonging time to death | 28 |
| To evaluate the effect of tozorakimab | Number of days alive and free of |
| versus placebo as add-on to SoC on | IMV/ECMO (WHO CPS score < 7) over 28 |
| ventilator use | day and 60 day period |
| | Number of days alive and ventilator free |
| | (WHO CPS score < 6) over 28 day and 60 |
| | day period |
| | Number of days alive and free of |
| | supplemental oxygen over 60 day period |
| To evaluate the effect of tozorakimab | Proportion of participants with ICU |
| versus placebo as add-on to SoC on ICU | admission or death by Day 60 |
| admissions | Proportion of participants with ICU |
| | admission or death by Day 28 |
| | Number or days alive and outside of ICU |
| | over 60 day period |
| To evaluate the effect of tozorakimab | Proportion of participants alive and |
| versus placebo as add-on to SoC on | discharged by Day 28 |
| duration of hospitalisation | Proportion of participants alive and |
| | discharged by Day 60 |
| | Time to discharge (time to WHO CPS score |
| | ≤3) |
| | Time to being off supplemental oxygen |
| To evaluate the effect of tozorakimab | WHO Clinical Progression Scale rank- |
| versus placebo as add-on to SoC on | based comparison |
| clinical status as assessed by the | |
| Investigator using WHO 10-category | |
| ordinal Clinical Progression Scale by Day | |
| 60 | |
| To evaluate the PK and immunogenicity of | PK: Serum tozorakimab concentrations |
| tozorakimab In participants with viral lung | Immunogenicity: Incidence of ADA |
| infection requiring supplemental oxygen | |
| To evaluate the use of baseline IL- | Baseline serum IL-33/sST2 relative to |
| 33/sST2 levels to predict treatment | primary endpoint |
| response with tozorakimab versus | |

^{a} Multiplicity controlled statistical analysis will also be conducted using the Full Analysis Set.
^{b} Relevant intercurrent events in addition to death include the receiving of additional treatments, changes to background treatments or changes in care setting.
^{c} All secondary endpoints, with the exception of PK, will be analysed in both the confirmed viral positive analysis set and full analysis set.
ADA = Anti-drug antibodies;
AE = Adverse event(s);
ECG = Electrocardiogram;
ED = Emergency department;
ECMO = Extracorporeal membrane oxygenation;
ER = Emergency room;
HRCU = Health care resource utilisation;
ICU = Intensive care unit;
IMV = Inter-module ventilation;
PK = Pharmacokinetic(s);
SAE = Serious adverse events(s);
SoC = Standard of care;
WHO = World Health Organization.

### Study Population

### Inclusion Criteria

Participants are eligible to be included in the study only if all of the following criteria apply: Age:
1. Adult participants ≥18 years old at the time of signing the ICF.

Type of Participant and Disease Characteristics:
2. Patients hospitalised with viral lung infection. Note: Suspected viral aetiology is acceptable to meet this criterion.
3. Hypoxaemia requiring treatment with supplemental O2, consistent with WHO Clinical Progression Scale for Disease Progression score of 5 and 6. Note: Hypoxemia is defined as SPO₂≤90% OR SPO₂≤92% AND one or both of the following:
   a. Radiographic infiltrates by Chest X-ray/CT scan compatible with viral lung infection per investigator judgement.
   b. Use of accessory muscles of respiration or respiratory rate >22/minute.
   Note: Patients receiving oxygen >6 L/min or non-invasive ventilation will be considered to have met this inclusion criterion regardless of SPO₂ levels. A documented pre-hospital SPO₂ (related to the episode) is acceptable, e.g., from the ambulance report.
4. ≤36 hours since admission to hospital.
5. ≤14 days since onset of respiratory viral infection symptoms.

### Exclusion Criteria

Participants are excluded from the study if any of the following criteria apply:

### Medical Conditions

1. Known fungal or parasitic lung infection, aspiration lung infection, lung abscess, or pulmonary sepsis. Bacterial co-infection is allowed, unless, in the opinion of the investigator, bacterial infection defines the severity of the participant's condition.
2. Hypoxaemia caused primarily by extrapulmonary insult (e.g., multiorgan failure, shock, or sepsis) or by lung injury of non-infective aetiology (eg, trauma, chemical injury, etc).
3. Ongoing or impending IMV/ECMO at randomisation.
4. Any comorbid condition that, in the opinion of the investigator, is likely to result in death within 3 months from randomisation.
5. Anticipated recovery and discharge from the hospital within 24 hours of randomisation.
6. Active tuberculosis defined as requiring current treatment.
7. Known unstable cardiovascular disease (e.g., unstable chronic heart failure NYHA III-IV, recent myocardial infarction or stroke within 3 months, or uncontrolled ventricular arrythmia) that in the investigator's judgement may put the participant at risk or negatively affect the outcome of the study.
8. Known absolute neutrophil count ≤1.0×109/L.
9. Known untreated HIV. Known history of active hepatitis B or C (treated and controlled hepatitis is allowed).
10. Known history of active severe inflammatory bowel disease or colitis (including Crohn disease or ulcerative colitis).
11. The following malignancies:
   a. Solid tumours with metastases (Stage IV).
   b. Lymphoma/leukaemia not in complete remission.
   c. Malignancies treated with chemotherapy and/or immunomodulatory drugs within the past 2 months.
12. Transplant patients at risk of organ rejection, or those on long-term immunosuppressive treatment for the transplant. Treatment with corticosteroids is allowed.
13. Any disorder that is not stable in the opinion of the investigator, including but not limited cardiovascular, gastrointestinal, hepatic, renal, neurological, musculoskeletal, infectious (including risk factors for viral lung infection), endocrine, metabolic, haematological, immune, psychiatric, or major physical impairment and could:
   affect the safety of the participant throughout the study,
   influence the findings of the study or their interpretation,
   impede the participant's ability to complete the entire duration of the study.

### Prior/Concomitant Therapy

14. Use of long-term oxygen therapy for pre-existing conditions.
15. Chronic treatment with TNF inhibitors, Janus kinase inhibitors or interferon-gamma. Wash-out period of 4 weeks or 5 half-lives (whichever is longer) is required prior to enrolment.
16. Current treatment with any investigational medication. Wash-out period of 4 weeks or half-lives (whichever is longer) is required prior to prior to enrolment.
17. Participants who have previously received tozorakimab.
18. Known history of:
   anaphylaxis to any other biologic therapy,
   severe reaction to any medication including biologic agents or human gamma globulin therapy,
   allergy or reaction to any component of the study intervention formulation.

Sequences

| SEQ ID | | |
|---|---|---|
| NO: | Description | Sequence |
| 1. | MEDI3506 VHCDR1 (amino | SYAMS |
| | acid) | |
| 2. | MEDI3506 VHCDR2 (amino | GISAIDQSTYYADSVKG |
| | acid) | |
| 3. | MEDI3506 VHCDR3 (amino | QKFMQLWGGGLRYPFGY |
| | acid) | |
| 4. | MEDI3506 VLCDR1 (amino | SGEGMGDKYAA |
| | acid) | |
| 5. | MEDI3506 VLCDR2 (amino | RDTKRPS |
| | acid) | |
| 6. | MEDI3506 VLCDR3 (amino | GVIQDNTGV |
| | acid) | |
| 7. | MEDI3506 VH Domain | EVQLLESGGGLVQPGGSLRLSCAASGFT |
| | (amino acid) | FSSYAMSWVRQAPGKGLEWVSGISAIDQ |
| | | STYYADSVKGRFTISRDNSKNTLYLQMN |
| | | SLRAEDTAVYYCARQKFMQLWGGGLRY |
| | | PFGYWGQGTMVTVSS |
| 8. | MEDI3506 VL Domain | SYVLTQPPSVSVSPGQTASITCSGEGMG |
| | (amino acid) | DKYAAWYQQKPGQSPVLVIYRDTKRPSG |
| | | IPERFSGSNSGNTATLTISGTQAMDEADY |
| | | YCGVIQDNTGVFGGGTKLTVL |
| 9 | MEDI3506 light chain | SYVLTQPPSVSVSPGQTASITCSGEGMG |
| | (amino acid) | DKYAAWYQQKPGQSPVLVIYRDTKRPSG |
| | | IPERFSGSNSGNTATLTISGTQAMDEADY |
| | | YCGVIQDNTGVFGGGTKLTVLGQPKAAP |
| | | SVTLFPPSSEELQANKATLVCLISDFYPG |
| | | AVTVAWKADSSPVKAGVETTTPSKQSNN |
| | | KYAASSYLSLTPEQWKSHRSYSCQVTHE |
| | | GSTVEKTVAPTECS |
| 10. | MEDI3506 heavy chain | EVQLLESGGGLVQPGGSLRLSCAASGFT |
| | (amino acid) | FSSYAMSWVRQAPGKGLEWVSGISAIDQ |
| | | STYYADSVKGRFTISRDNSKNTLYLQMN |
| | | SLRAEDTAVYYCARQKFMQLWGGGLRY |
| | | PFGYWGQGTMVTVSSASTKGPSVFPLA |
| | | PSSKSTSGGTAALGCLVKDYFPEPVTVS |
| | | WNSGALTSGVHTFPAVLQSSGLYSLSSV |
| | | VTVPSSSLGTQTYICNVNHKPSNTKVDK |
| | | RVEPKSCDKTHTCPPCPAPELLGGPSVF |
| | | LFPPKPKDTLMISRTPEVTCVVVDVSHED |
| | | PEVKFNWYVDGVEVHNAKTKPREEQYN |
| | | STYRVVSVLTVLHQDWLNGKEYKCKVSN |
| | | KALPAPIEKTISKAKGQPREPQVYTLPPS |
| | | REEMTKNQVSLTCLVKGFYPSDIAVEWE |
| | | SNGQPENNYKTTPPVLDSDGSFFLYSKL |
| | | TVDKSRWQQGNVFSCSVMHEALHNHYT |
| | | QKSLSLSPGK |
| 11. | IL-33 full length protein | MKPKMKYSTNKISTAKWKNTASKALCFK |
| | | LGKSQQKAKEVCPMYFMKLRSGLMIKKE |
| | | ACYFRRETTKRPSLKTGRKHKRHLVLAA |
| | | CQQQSTVECFAFGISGVQKYTRALHDSSI |
| | | TGISPITEYLASLSTYNDQSITFALEDESY |
| | | EIYVEDLKKDEKKDKVLLSYYESQHPSNE |
| | | SGDGVDGKMLMVTLSPTKDFWLHANNK |
| | | EHSVELHKCEKPLPDQAFFVLHNMHSNC |
| | | VSFECKTDPGVFIGVKDNHLALIKVDSSE |
| | | NLCT ENILFKLSET |

1. A method of treating or preventing acute respiratory distress syndrome (ARDS) in a subject suffering from or at risk of developing ARDS, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

2. A method of treating or preventing acute respiratory failure (ARF) in a subject suffering from or at risk of developing ARF, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

3. A method of treatment to reduce the risk of a subject requiring invasive mechanical ventilation (IMV) or extracorporeal membrane oxygenation (ECMO), wherein the subject has or is at risk of developing ARDS and/or ARF, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

4. A method of treatment to reduce the risk of a subject with, or at risk of developing, ARDS and/or ARF requiring admission to an intensive care unit (ICU), the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

5. A method of treatment for reducing the duration of hospitalisation of a subject with, or at risk of developing, ARDS and/or ARF, the method comprising administering to the subject a dose of 250 to 350 mg of an anti-IL-33 antibody, wherein the antibody comprises:
((a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

6. A method of treatment of a subject with, or at risk of developing, ARDS and/or ARF and who requires supplemental oxygen therapy, which treatment reduces the required duration of the supplemental oxygen therapy, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

7. The method of any one of claims 1 to 6, wherein the anti-IL-33 antibody comprises a heavy chain variable region comprising the sequence of SEQ ID NO: 7, or an amino acid sequence with at least 80% sequence identity thereto; and
a light chain variable region comprising the sequence of SEQ ID NO: 8, or an amino acid sequence with at least 80% sequence identity thereto.

8. The method of claim 7, wherein the anti-IL-33 antibody is tozorakimab.

9. The method of any one of claims 1 to 8, wherein the anti-IL-33 antibody is administered to the subject intravenously.

10. The method of any one of claims 1 to 9, wherein the dose of the anti-IL-33 antibody is 300 mg.

11. The method of any one of claims 1 to 10, wherein a single dose of the anti-IL-33 antibody, is administered to the subject.

12. The method of any one of claims 1 to 11, wherein the subject has or is suspected of having a viral lung infection.

13. The method of any one of claims 1 to 12, wherein the subject has or is suspected of having a viral lower respiratory tract infection or disease.

14. The method of claim 12 or 13, wherein the viral lung infection is caused by a coronavirus, such as SARS-CoV-2.

15. The method of claim 12 or 13, wherein the viral lung infection is not a SARS-CoV-2 infection.

16. The method of claim 12 or 13, wherein the viral lung infection is caused by influenza virus, such as influenza virus A or influenza virus B.

17. The method of claim 12 or 13, wherein the viral lung infection is caused by RSV.

18. The method of claim 12 or 13, wherein the viral lung infection is caused by human metapneumovirus (HMPV).

19. The method of any one of claims 1 to 18, wherein the subject is hospitalised.

20. The method of claim 19, wherein the subject is hospitalised due to the viral lung infection or suspected viral lung infection.

21. The method of claim 19 or claim 20, wherein the subject is hospitalised due to a viral lower respiratory tract infection or disease, or a suspected viral lower respiratory tract infection or disease.

22. The method of any one of claims 19, 20 or 21, wherein the anti-IL-33 antibody is administered to the subject up to about 36 hours after the subject is admitted to hospital.

23. The method of any one of claims 12 to 22, wherein the anti-IL-33 antibody is administered to the subject up to about 14 days after the onset of symptoms of a viral lung infection in the subject.

24. The method of any one of claims 1 to 23, wherein the subject requires supplemental oxygen or ventilation.

25. The method of any one of claims 1 to 24, wherein the subject has hypoxemia and requires supplemental oxygen.

26. The method of any one of claims 1 to 25, wherein the subject has a viral lower respiratory tract infection or disease, is hospitalized, and requires supplemental oxygen or ventilation.

27. The method of claim 24 or 25, wherein the subject:
(i) has an SpO₂≤94% on room air;
(ii) had an SpO₂≤94% prior to initiation of oxygen therapy; and/or
(iii) is receiving >6 L/min supplemental oxygen and/or non-invasive ventilation.

28. The method of claim 24 or 25, wherein the subject has an:
(i) SpO2≤90% or
(ii) SpO2≤92% and one or both of the following: radiographic infiltrates by Chest X-ray/CT scan compatible with viral lung infection per investigator judgement or use of accessory muscles of respiration or respiratory rate >22/minute.

29. The method of any one of claims 1 to 28, wherein the anti-IL-33 antibody is tozorakimab, and is administered in a solution comprising:
(a) 20 mM±10% L-histidine/L-histidine-hydrochloride;
(b) 220 mM±10% L-arginine hydrochloride; and
(c) 0.03%±10% w/v polysorbate 80;
and which has a pH of 5.5;
suitably wherein the solution comprises 150 mg/ml tozorakimab.

30. The method of any one of claims 1 to 29, wherein after administration of the anti-IL-33 antibody, the subject is administered a saline flush, optionally wherein the saline flush comprises 0.9%±10% w/v NaCl and has a pH of 5.5, optionally wherein the saline flush is a 5 ml saline flush.

31. The method of any one of claims 1 to 30, wherein the treatment reduces the risk of the subject developing ARDS or pneumonia, particularly within 28 days or 60 days following administration of the anti-IL-33 antibody.

32. The method of any one of claims 1 to 31, wherein the treatment reduces the risk of the subject dying within 28 or 60 days following administration of the anti-IL-33 antibody, in particular reducing the risk of the subject dying from ARDS in said timeframe.

33. The method of any one of claims 1 to 32, wherein the treatment reduces the risk of the subject requiring admission to ICU or requiring invasive mechanical ventilation (IMV) or extracorporeal membrane oxygenation (ECMO) within 28 or 60 days following administration of the anti-IL-33 antibody; and/or reduces the duration of ICU admission, IMV or ECMO required within 28 or 60 days following administration of the anti-IL-33 antibody.

34. The method of any one of claims 1 to 33, wherein the treatment increases the number of days the subject is alive and outside ICU within 60 days following administration of the anti-IL-33 antibody, and/or increases the number of days the subject is alive and does not require supplementary oxygen within 60 days following administration of the anti-IL-33 antibody.

35. The method of any one of claims 1 to 34, wherein the treatment reduces the duration of supplemental oxygen administration required by the subject within 28 or 60 days following administration of the anti-IL-33 antibody.

36. The method of any one of claims 1 to 35, wherein the treatment reduces the required length of hospitalisation of the subject, and/or increases the chance of the subject being discharged from hospital alive within 28 or 60 days following administration of the anti-IL-33 antibody.

37. The method of any one of claims 1 to 36, wherein the treatment reduces the risk of hospital readmission of the subject after discharge within 28 or 60 days following administration of the anti-IL-33 antibody.

38. The method of any one of claims 1 to 37, wherein the treatment reduces or prevents disease progression as defined by the WHO 10-point clinical progression scale for COVID-19, or causes an improvement in the condition of the subject as defined by said clinical progression scale,
for example, wherein the treatment increases the chance of the subject having a score less than 7, 6, 5, 4 or 3 as defined by the WHO 10-point clinical progression scale for COVID-19 at day 28 or 60 following administration of the anti-IL-33 antibody, or causes the subject to reach a score of less than 7, 6, 5, 4 or 3 according to said scale more quickly.

39. An anti-IL-33 antibody for use in the treatment or prevention of ARDS or ARF in a subject, wherein the antibody, treatment or prevention, ARDS, ARF and/or subject are as defined in any one of claims 1 to 38.

40. Use of an anti-IL-33 antibody in the manufacture of a medicament for the treatment or prevention of ARDS or ARF in a subject, wherein the antibody, treatment or prevention, ARDS, ARF and/or subject are as defined in any one of claims 1 to 38.

41. A pharmaceutical composition comprising an anti-IL-33 antibody for use in the treatment or prevention of ARDS or ARF in a subject, wherein the antibody, treatment or prevention, ARDS, ARF and/or subject are as defined in any one of claims 1 to 38.

42. A method of treatment for preventing or reducing the risk of acute respiratory failure or ARDS in a subject hospitalised with a viral lung infection or suspected viral lung infection, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

43. A method of treating a subject hospitalised with a viral lung infection or suspected viral lung infection, the method comprising administering to the subject a dose of 250 to 350 mg of an anti-IL-33 antibody, wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

44. The method of claim 43, wherein the subject requires the use of supplemental oxygen.

45. The method of claim 42 or 43, wherein the anti-IL-33 antibody, administration, viral lung infection, subject and/or treatment is as defined in any one of claims 7 to 11, 14 to 18 or 22 to 37.

46. An anti-IL-33 antibody for use in the treatment or prevention of respiratory failure in a subject hospitalised with a viral lung infection or suspected viral lung infection, wherein said treatment comprises administering to the subject a dose of 250 to 350 mg of the anti-IL-33 antibody, and wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

47. An anti-IL-33 antibody for use in the treatment of a viral lung infection or suspected viral lung infection in a subject, wherein the subject is hospitalised with the viral lung infection or suspected viral lung infection, wherein said treatment comprises administering to the subject a dose of 250 to 350 mg of the anti-IL-33 antibody, and wherein the antibody comprises:
(a) a heavy chain variable region comprising a VHCDR1 having the sequence of SEQ ID NO: 1, a VHCDR2 having the sequence of SEQ ID NO: 2, and a VHCDR3 having the sequence of SEQ ID NO: 3; and
(b) a light chain variable region comprising a VLCDR1 having the sequence of SEQ ID NO: 4, a VLCDR2 having the sequence of SEQ ID NO: 5, and a VLCDR3 having the sequence of SEQ ID NO: 6.

48. The anti-IL-33 antibody, for use according to claim 42 or 43, wherein the anti-IL-33 antibody, administration, viral lung infection, subject and/or treatment is as defined in any one of claims 7 to 11, 14 to 18 or 22 to 37.
